(19) United States
(12) Patent Application Publication
(10) Pub. No.: US 2018/0318139 A1
(43) Pub. Date: Nov. 8, 2018

(54) MEDICAL PLASTER

(71) Applicants: Petros Savrou, Hamburg (DE); Andre Piontek, Hamburg (DE)

(72) Inventor: Petros STAVROU, Hamburg (DE)

(21) Appl. No.: 15/759,978

(22) PCT Filed: Jul. 13, 2016
(86) PCT No.: PCT/DE2016/100314

§ 371 Date: Mar. 30, 2018

(30) Foreign Application Priority Data
Sep. 15, 2015 (DE) 20 2015 006 520.1
Nov. 3, 2015 (DE) 10 2015 118 780.9

(51) Int. Cl. A61F 13/02; A61F 13/00; A61F 7/02

(52) U.S. Cl. CPC A61F 13/0206; A61F 13/0246; A61F 2007/0001; A61F 7/02; A61F 13/00063

(57) ABSTRACT

The invention relates to a medical plaster (1) with a layer (2) of textile fabric or plastic, an adhesive layer (5), and an absorbent dressing layer (3). To permit individual dosing of an active substance and/or a physical cooling effect, it is proposed that the fabric or plastic layer (2) has an opening (4) in the area of the dressing layer (3), such that the dressing layer (3) is exposed and accessible in the area of the opening (4). The invention moreover comprises a medical kit (100) with the plaster, and a hood that has the plaster (1) as an integral part.

### FIELD OF THE INVENTION

The invention relates to a medical plaster having a layer of textile fabric or plastic, an adhesive layer and an absorbent dressing layer which is attached to the fabric layer or plastic layer.

### BACKGROUND OF THE INVENTION

Plasters of the aforementioned type, for example, rapid wound dressings, are used for covering wounds and minor injuries and accelerating wound healing. The plasters may contain an antiseptic or some other active pharmaceutical substances. One disadvantage is that the amount of active ingredient is predetermined and cannot be dosed individually. Furthermore, the active ingredients can be absorbed through the skin and cause adverse effects.

In addition, there are so-called cold plasters or cooling plasters, which are used on inflamed locations without any external injuries, such as compressions, contusions, strains and the like. Such a plaster is known from the Unexamined German Patent Application DE 38 32 451 A1. These plasters contain active ingredients such as menthol, which are supposed to relieve symptoms by cooling. In fact, however, the active ingredients merely bind to the cold receptor TRP melastatin 8, so that although a cooling effect is perceived subjectively, the temperature of the skin and/or body is not reduced physically. However, lowering the temperature physically has anti-inflammatory and analgesic effects as well as a detumescent effect on injuries or surgical procedures.

### SUMMARY OF THE INVENTION

The present invention was developed against the background of the prior art as described above. The object of the invention is to propose a medical plaster having a layer of textile fabric or plastic, an adhesive layer and an absorbent dressing layer, which is attached to the fabric layer or plastic layer, so that the disadvantages mentioned above can be avoided.

This object is achieved in that the fabric layer or plastic layer has an opening in the area of the dressing layer so that the dressing layer is exposed and accessible in the area of the opening. Therefore an active ingredient can be applied to the absorbent dressing layer, which forms a reservoir of active ingredient. The advantage thereby achieved consists of the fact that neither the type of active ingredient nor the quantity of active ingredient is predetermined but instead they can be selected as needed by adding the substance. This allows an individualized treatment, based on the individual case. Another advantage is that the active ingredient can be re-dosed without having to remove the plaster. A substantial additional advantage is that the plaster can be used as a physically effective cooling plaster. A liquid in the dressing layer can evaporate through the opening and thereby create the cooling effect of evaporation. The temperature of the inflamed site is actually lowered in this way, and an anti-inflammatory effect is created, which is not achieved with traditional menthol plasters/dressings. The plaster according to the invention can be used in a variety of ways, for example, on arthritis, hematomas, swelling, insect bites, etc. Use is not limited to humans but instead this plaster may also be used in veterinary medicine. The term “plaster” should not limit the size and shape of the plaster, and instead the plaster according to the invention may have any required size and shape. The dressing layer is attached to the layer of textile fabric by the adhesive layer. The adhesive layer may, but need not necessarily go beyond the area of the dressing layer, so that the plaster can be attached to the skin. The dressing layer may be a layer of gauze or nonwoven for example.

Advantageous embodiments of the invention with additional features are described below.

The dressing layer of the plaster may have at least one active ingredient. The term “active ingredient” here comprises physically active and/or pharmaceutically active substances. For example, in the simplest case, the active ingredient may be water or an alcohol or a mixture that evaporates and thereby generates cold by physical means. It may also be an active ingredient which generates and/or gives off heat. The active ingredient may also be an antiseptic or an analgesic agent, an antibiotic or the like. The effect of pharmaceutical substances may be manifested topically or transdermally. A combination of different active ingredients is also possible.

In one special embodiment as a cooling plaster, the dressing layer contains an aqueous menthol solution. A combined effect can be achieved in this way by the receptor binding of the menthol and also by the physical reduction in temperature, which is brought about by evaporation of the menthol solution.

A covering closure which preferably has pores that are permeable for gaseous and/or volatile substances may be provided for the opening. In this way the dressing layer can be protected from soiling and the plaster can nevertheless have all of the aforementioned advantages and effects.

The invention also comprises a hood for the head which may be designed approximately in the manner of a surgical hood, which has an integral medical plaster on the top side having the features described above. For example, the hood may be used for injuries or after surgical procedures, for example, after a hair transplant. The transplanted areas of skin can then be cooled and/or supplied with medication.

The invention also comprises a medical kit which includes a medical plaster having the features described above or the aforementioned hood or both parts. In addition, a container with at least one active ingredient is provided to apply the active ingredient(s) to the plaster or the hood with the plaster.

The medical kit may have in particular an applicator for applying the active ingredient to the plaster or the container may be designed as the applicator.

The applicator may be designed as a pipette for pipetting the active ingredient onto the plaster.

The active ingredient in the container or applicator may preferably be an aqueous menthol solution. As soon as the menthol solution has evaporated, it can be resupplied with the pipette.

### BRIEF DESCRIPTION OF THE DRAWINGS

The figures show in detail:

FIG. 1 a schematic top view of a plaster;

FIG. 2 a medical kit consisting of a pipette, active ingredient and the plaster from FIG. 1 in a vertical section; and

FIG. 3 a perspective view of a hood for the head with a plaster from FIG. 1.

Parts having the same function are labeled with the same reference numerals.

### DETAILED DESCRIPTION OF PREFERRED EMBODIMENTS

Preferred embodiments of the invention are described in detail below with reference to the drawings, such that additional advantageous features can be derived from the diagrams in the figures.

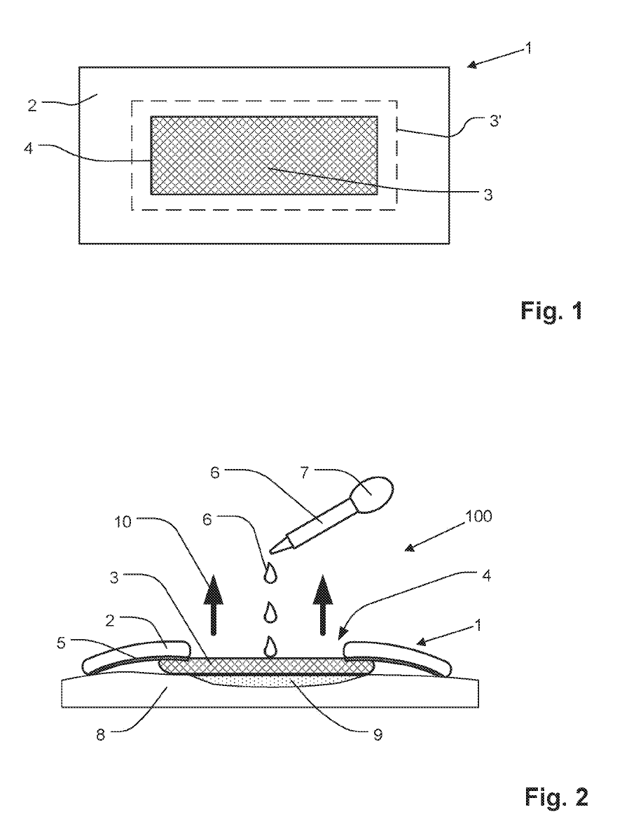

FIG. 1 shows a top view of the plaster 1. It has an upper plastic layer 2. The plastic layer 2 has an opening 4. An absorbent gauze layer 3, which is slightly larger than the opening 4, is arranged beneath the plastic layer 2. This is illustrated by the dotted lines 3′. The gauze layer 3 is glued to the plastic layer 2 with its overlapping edge region 3′.

FIG. 2 shows a medical kit 100 comprised of a pipette 7, active ingredient 6 and plaster 1 from FIG. 1 in a vertical section. The gauze layer 3 is attached to the upper plastic layer 2 by means of the adhesive layer 5. FIG. 2 also shows an area of skin 8 with an inflamed location 9. The plaster 1 is glued to the area of skin 8 with the lower adhesive layer 5 so that the inflamed location 9 is covered by the absorbent gauze layer 3.

An opening 4 is provided within the upper plastic layer 2, so the gauze layer 3 is accessible from the top side. An aqueous menthol solution 6 can be dripped onto the absorbent gauze layer 3 by means of the pipette 7 through the opening 4. The menthol solution 6 can evaporate through the opening 4 (represented as arrow 10) and this causes heat to be withdrawn from the inflamed location 9. This lowers the skin temperature and has an ameliorating, detumescent and anti-inflammatory effect. The effect is further enhanced by the presence of menthol.

When the aqueous menthol solution 6 evaporates, more can be added by pipette without having to replace the plaster 1.

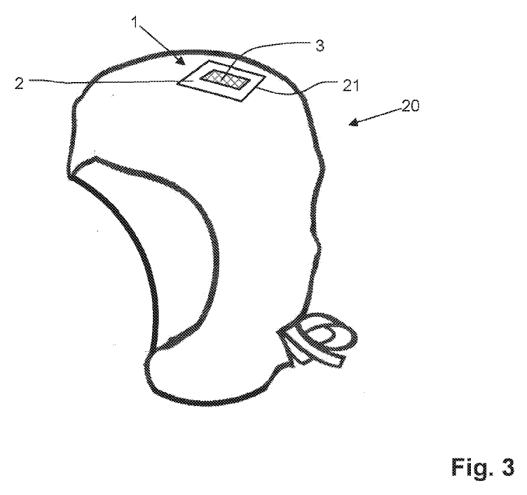

FIG. 3 shows a perspective view of a hood 20 for the head with an integrated plaster 1. The hood 20 may be a conventional surgical hood, but it has an opening 21 at a location provided for it. In FIG. 3, the opening 21 is arranged on the top side of the hood 20. A medical patch 1 as described in FIGS. 1 and 2 is arranged above this opening 21. The opening 21 is slightly smaller than the plaster 1, so that the latter is attached by adhesive bonding to the edges of the opening 21 (not shown here).

For example, the hood 20 may be used on injuries or after surgical procedures, for example, after a hair transplant. The transplanted areas of skin can then be cooled and/or supplied with medication.

### LIST OF REFERENCE NUMERALS

1. plaster
2. plastic layer
3. gauze
4. opening
5. adhesive layer
6. active ingredient solution
7. pipette
8. skin
9. inflammation
10. evaporation
20. hood
21. opening
100. medical kit

1. A medical plaster comprising a layer of textile fabric or plastic, an adhesive layer and an absorbent dressing layer which is attached to the layer of textile fabric or plastic wherein the layer of textile fabric or plastic includes an opening in the area of the dressing layer so that the dressing layer is exposed and accessible in the area of the opening.

2. The medical plaster according to claim 1, wherein the absorbent dressing layer comprises at least one active ingredient.

3. The medical plaster according to claim 1 further comprising a covering closure for the opening.

4. A hood for a head comprising a medical plaster in accordance with claim 1.

5. A medical kit comprising a hood according to claim 4 and a container having at least one active ingredient.

6. The medical kit according to claim 5 further comprising an applicator for applying the active ingredient to the plaster.

7. The medical kit according to claim 6 wherein the applicator comprises a pipette for pipetting the active ingredient onto the plaster.

8. The medical kit according to claim 5 wherein the active ingredient comprises an aqueous menthol solution.

9. The medical plaster according to claim 2, wherein the active ingredient comprises an aqueous menthol solution.

10. The medical kit according to claim 6 wherein the container is also an applicator.

11. The medical plaster according to claim 2, wherein the covering closure comprises pores that are permeable to gaseous andior volatile substances.

12. A medical kit comprising a medical plaster according to claim 1 and a container having at least one active ingredient.

13. The medical kit according to claim 12 further comprising an applicator for applying the active ingredient to the plaster.

14. The medical kit according to claim 13 wherein the applicator comprises a pipette far pipetting the active ingredient onto the plaster.

15. The medical kit according to claim 12 wherein the active ingredient comprises an aqueous menthol solution.

16. The medical kit according to claim 13 wherein the container is also an applicator.
